(19) United States
(12) Patent Application Publication
(10) Pub. No.: US 2005/0188994 A1
(43) Pub. Date: Sep. 1, 2005

(54) Male genital sheart for providing greater protection against sexually transmitted diseases enhanced pleasure, and improved performance

(76) Inventor: Carol Wasserman Star, Honolulu, HI (US)

Correspondence Address: Carol Wasserman Star, 2333 Kapiolani Bld #1813, Honolulu, HI, 96826, US

(21) Appl. No.: 10/487,672

(22) PCT Filed: Dec. 28, 2002
(86) PCT No.: PCT/US02/41907

§ 371 Date: Feb. 24, 2004

Related U.S. Application Data
(60) Provisional application No. 60/342,896, filed on Dec. 28, 2001.

(51) Int. Cl. A61F 6/04

(52) U.S. Cl. 128/844

(57) ABSTRACT

A realiable cover that is roughly tubular and that has two open ends that applies to the purposes of disease prevention and enhanced pleasure. A sheath covers the entire scrotum and extends partly up the pcnile shaft. A restraining strap, optionally attached and with fixable ends, is wrapped around the periphery of the proximal open end, guided by a finishing lip or band, in order to secure the scrotum within the scrotal sac and also to anchor the sheath during use. In a modified form there is wider proximal opening for more comfort of application.

This application claims priority of the previous provisional application U.S. 60/342,896 filed Dec. 28, 2001

This patent is related to my prior patent U.S. Pat. No. 6,209,543, Condom providing enhanced protection against sexually transmitted diseases, issued Apr. 4, 2001 which is hereby incorporated by reference. The scrotal condoms of U.S. Pat. No. 6,209,543 are one piece with an open proximal end and a distal closed end, covers the entire shaft, may require manufacturing in different sizes and is atypical of the traditional shaft condom. This new patent is open at two ends and does not cover the entire shaft of the penis, thus it may be used as an accessory with an FDA approved and publically accepted over the counter condom of the user's choice that may be unrolled to needed length or used independently for sensuality, scrotal firmness, and prolonged erection without penile restriction of the shaft portion while providing protection against certain STDs.

### FIELD OF THE INVENTION

The present invention relates to an improved genital prophylactic intended to effectively protect sexually active men and women at risk of contracting one of the twenty or more known sexually transmitted diseases (STDs) prevalent today in populations throughout the U.S. and worldwide. More specifically, it relates to a roughly tubular, non-continuous shaft that covers the lower part of the penile shaft and scrotum of the male thus minimizing skin-to-skin contact with the vaginal and labial regions of the female. It uses a restraining strap and a restrictive band to minimize slippage, thus providing comprehensive and reliable protection against disease. The firm fit around the base of the scrotum and the optional restraining strap adds pressure to the Pubococcygeus muscle, thereby keeping blood in the shaft portion which enhances arousal, prolongs erectness, adds firmness, and durability. The scrotal sac keeps the testes firm, prevents sagging scrotal sac and minimizes swaying movement, thus increasing contact with the female external genitalia.

### BACKGROUND OF THE INVENTION

Sexually transmitted diseases (STDs) continue to spread at an alarming rate throughout the U.S. population. In fact, it is estimated that 12 million people in the U.S. acquire some form of STD each year. Overall, about one in four Americans has a STD. This category includes such conditions as herpes, human papilloma virus, uterine cancers, penile cancers, syphilis, gonorrhea, trichomoniasis, chlamydia, infertility and HIV (AIDS), among others. STDs are most commonly spread when sexual partners engage in unprotected sex, that is, the infected genitalia (and surrounding area) of one partner comes into contact with the genitalia (and surrounding area) of another partner, thus transferring the infection. Although most STDs respond to some form of treatment, certain sexually transmitted pathogens cause cervical, liver, and other cancers, while infections in pregnant women can cause spontaneous abortion, stillbirth, pre-term delivery, and illness among infants.

The objects of this invention are to provide sexual partners with effective protection against STDs, sexual dysfunction and increase pleasure for both male and female partners. This has been accomplished by fashioning a genital prophylactic with a scrotal and partial penile covering with an optionally removable restraining strap. Together, the sheath covering and strap are intended to provide improved prophylactic protection, when used with a traditional condom, as compared with a traditional continuous shaft condom that does not include scrotal coverage or restraining capability and may expose the penile shaft and vagina during intercourse. The scrotal and penile covering also provides more comprehensive protection against disease when used with a regular condom, by preventing contact between the scrotum and the labia, and between the scrotum and the anal region. The restraining strap and band provide more reliable protection in that it anchors the prophylactic, thus safeguarding against the genital cover slipping partly or completely off during intercourse, a consequence known to occur with traditional, continuous shaft condoms used by most condom users throughout the world today.

To further increase protection against slippage, the shaft portion of the sheath includes an optional restrictive band. The restrictive strap and band act like a cock ring to hold blood in the erect penis, thus prolonging erection, increasing durability and hardness. The restrictive strap and band, which is secured under the scroturn puts pressure on the pubococcygeus muscle, which is used in exercises and tantric sexual practices designed to prolong and control ejaculation while increasing the ability for multiple orgasms. This genital cover may be worn as a sexual novelty and accessory without the use of a regular condom for enhanced pleasure and performance, when both partners are not concerned about prevention of pregnancy and disease prevention. This feature may also be desired for older men or men with drooping or large hanging testes.

This invention is an advancement over prior art patents of a partially similar construction in many important ways: (1) Construction of the scrotal and partial penile covering for added protection without skin-to-skin genital or anal contact; (2) The option of using the male genital sheath with the user's condom of choice; (3) Firming the scrotum for more compact contact and uplifting of sagging and large testes; (4) Added protection for those that may be concerned about scrotal skin diseases or have scrotal outbreaks and may or may not want to use a traditional condom; (5) A restrictive band or optional band which adds pressure on the Pubococcygeus muscle for hardness and durability; (6) Compact, smaller, and less bulky than the one piece scrotal condoms and has pocket size portability; (7) As an accessory; the public continues to use the regular shaft condom that they are used to with the sheath as a simple addition (8) Novelty of a new product design to increase use; (9) As an accessory the condom industry could add this to their product line without making traditional shaft condoms seem outdated or inferior; (10) On the factory dipping line, the tanks would not need to be changed, since the standard tank depth works for manufacturing sheaths, but may be a technical problem with one-piece scrotal condoms; (11) The sheath and traditional condom may be rolled and made more compact for packaging.

For example, U.S. Pat. No. 6,209,543 and U.S. Pat. No. 5,318,042 and U.S. Pat No. 5,070,890 have the disadvantages of being of one piece covering shaft and sac. This adds additional torque, which may be a problem with breakage for men who have large penile shafts and large testes. The shaft portion may be too long for other users who have small penile members, which may create air pockets, noise and breakage. The one-piece scrotal condoms may need to be made in various shaft sizes, which complicates manufacturing. These require educating users to select the appropriate size, small, medium, and large, which could become a psychological issue. Those that purchase condoms for different partners may have a problem of size selection and use.

U.S. Pat. No. 4,966,165 is a unisex condom designed like an undergarment panty, is very atypical from the tubular shaft condoms. It covers a much larger part of the external body and is bulky to wear or carry in a wallet.

For example, U.S. Pat. No. 4,781,709 includes a large water-impervious sheet that extends upward to cover the male navel. It is very atypical of the tubular shaft condoms. It is very bulky and much larger than normal condoms that can fit into a wallet for portability.

For example, U.S. Pat. No. 5,070,890 and U.S. Pat. No. 5,314,447 include a scrotal sac in each embodiment, but the sac opening has the disadvantages of not being substantially wider than the shaft and of not being fashioned from a pliant construction, hindering admittance of the scrotum into the scrotal sac. And since condom usage is often dependent on convenience and comfort, this limitation could discourage usage, possibly during a sexual encounter where usage is necessary to prevent disease and/or pregnancy.

Likewise, The patents U.S. Pat. No. 5,318,042 and U.S. Pat No. 5,070,890 also have a limited access and rigid construction relative to the scrotal sac. Other than U.S. Pat. No. 6,209,543 the other patents do not provide a restraining mechanism to prevent the condom from slipping partly or completely off during sexual intercourse as does the present invention.

U.S. Pat. No. 4,354,494 does provide a restraining strap that fits over the scrotum. However, it is unclear if this strap would remain secure during sexual intercourse and if tension from this strap would cause discomfort to the testes, whereas the restraining strap of the present invention is designed to provide a comfortable, secure fit. Also significant, this patent does not provide a coverage over the scrotum. Therefore, it is unable to provide the same level of protection as the present invention.

U.S Pat. No. 5,111,831 provides a band around the scrotum for sensual purposes, but has a hole so the scrotum is uncovered. However, it does not have added protective covering over the scrotum or the added firmness by holding the scrotum in a sac.

### SUMMARY OF THE INVENTION

The prophylactic of the present invention provides an improved safety against STDs, the device comprising a roughly tubular, non-continuous shaft that is roughly cylindrical and that covers the lower portion of the shaft of the penis, and the scrotum. When used in conjunction with a regular shaft condom, it prevents skin-to-skin contact of the penile and scrotal areas with the vagina and labial regions. It also protects homosexual and heterosexual partners engaged in anal genital contact.

### BRIEF DESCRIPTION OF THE DRAWINGS

FIG. 1 is a side view of a fitted version of the present invention, shown in an unfastened state.

FIG. 2 is a proximal end view of the fitted sheath FIG. 1 in the unfastened state.

FIG. 3 is a side view of the fitted sheath FIG. 1, shown in a fastened state.

FIG. 4 is a proximal end view of the fitted sheath FIG. 3 in the fastened state.

FIG. 5 is a cross section of the fitted sheath of FIG. 1, shown in a rolled state for storage or application.

FIG. 6 is a side view of a semi-fitted version of the sheath of the present invention, shown in an unfastened state.

FIG. 7 is a proximal end view of the semi-fitted sheath of FIG. 6 in the unfastened state.

FIG. 8 is a side view of the semi-fitted sheath of FIG. 6, shown in a fastened state.

FIG. 9 is a proximal end view of the semi-fitted sheath of FIG. 8 in the fastened state.

FIG. 10 is a cross section of the semi-fitted sheath of FIG. 6, shown in a rolled state for storage or application.

### DETAILED DESCRIPTION OF THE INVENTION

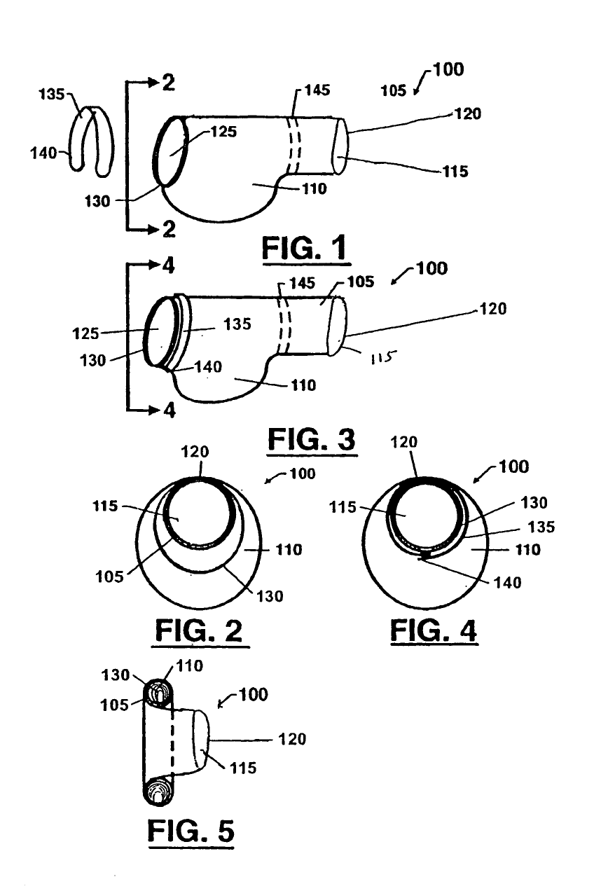

FIGS. 1 through 5 illustrate a first preferred embodiment of the present invention, which is a fitted version of a sheath 100 configured to cover a portion of user's penis and entire scrotum. FIG. 1 is a side view of the fitted sheath 100 in an unfastened state. FIG. 2 is a proximal end view of the fitted sheath 100 of FIG. 1 in the unfastened state. The sheath 100 is constructed with a roughly tubular shaft 105 that is attached at its proximal end to a scrotal sac 110. The scrotal sac 110 is asymmetrical with the tubular shaft 105 in keeping with the normal anatomical configuration of the male genitalia. When properly fitted, the tubular shaft 105 covers the proximal portion of the penis and the scrotal sac 110 covers the entire scrotum. The sheath 100 has one open end 115 at the distal end of the tubular shaft 105, and one open end 125 at the proximal end of the scrotal sac 110 for admitting the user's penis and the scrotum.

The scrotal sac 110 is sized to loosely and comfortably fit the user's scrotum and testicles without discomfort or constriction. The proximal open end 125 of the sheath 100 is sized to easily and comfortably admit the user's scrotum and testicles into the scrotal sac 110 without discomfort or constriction. In this embodiment, the proximal open end 125 of the fitted sheath 100 is preferably somewhat smaller than the maximum diameter of the scrotal sac 110, but larger than the diameter of the tubular shaft 105. The periphery of the proximal open end 125 is preferably finished with a lip or band 130 that is distinctly rounded and extends slightly above the surface of the tubular shaft 105 and that provides a useful gripping surface for helping to apply the sheath 100 over the surface of the penis. In one preferred embodiment, the lip or band 130 around the proximal open end 125 of the scrotal sac 110 is approximately 1 mm thick. The periphery of the distal open end 120 is preferably finished with a lip or band 150 that is distinctly rounded and extends slightly above the surface of the tubular shaft 105 and that provides a useful gripping surface for helping to apply the sheath 100 over the surface of the penis and keep the traditional shaft condom overlapped with the sheath. In one preferred embodiment, the lip or band 130 around the proximal open end 125 of the scrotal sac 110 is approximately 1 mm thick.

In one preferred embodiment, the lip or band 150 around the distal open end 125 of the penile shaft 120 is approximately 1 mm thick.

Preferably, the condom 100 is made of a highly flexible and elastic polymer or elastomer. Suitable materials include, but are not limited to latex, polyurethane and other natural and synthetic rubbers. Preferably, the tubular shaft 105 and the scrotal sac 110 of the sheath 100 are manufactured together in one piece, such as by dip molding, out of a single material. Alternatively, however, the tubular shaft 105 and the scrotal sac 110 may be made of different materials and joined together, for instance by adhesive bonding or by heat welding. Additionally or alternatively, natural membranes or textile fabrics may be used in the construction of the tubular shaft 105 and/or scrotal sac 110 of the sheath 100. Additionally or alternatively the tubular shaft 105 and the scrotal sac 110 may have perforations, meshing or slits to add ventilation.

The fitted sheath 100 also includes a restraining strap 135 that, when fastened, secures the scrotum within the scrotal sac 110 and effectively anchors the cover 100 during use. The restraining strap 135 is preferably constructed of a relatively flat elastic material and has fixable ends 140. The fixable ends 140 of the restraining strap 135 include a fastener, such as corresponding halves of a VELCRO hook-and-loop fastener strip or a similarly effective fastener or an adhesive. In this illustrative embodiment, the restraining strap 135 is shown as separate from the sheath 100. Optionally, however, the restraining strap 135 may be permanently or removably attached to the sheath 100 near [symbol] the proximal open end 125. The restraining strap 135 is preferably sized to fasten the open end 125 of the scrotal sac 110 snugly and comfortably around the user proximal to the scrotum. In one preferred embodiment, the restraining strap 135 is approximately 1 cm wide, 10 cm long, and 1 mm thick. The lip or band 130 around the open end 125 of the scrotal sac 110 serves as a stopper ring to prevent the fastened restraining strap 135 from slipping off of the sheath 100.

For additional protection against slippage, the sheath 100 may optionally include a restrictive band 145, indicated by dashed lines in FIGS. 1 and 3, around the periphery of the tubular shaft 105. The restrictive band 145 is preferably located at the proximal end of the tubular shaft 105 where it joins to the scrotal sac 110. The restrictive band 145 may be formed by a slight thickening of the wall of the tubular shaft 105 and/or by a slight reduction in the internal diameter of the tubular shaft 105.

FIG. 3 illustrates the sheath 100 of FIG. 1 fastened and shows the restraining strap 135 properly positioned just distal to the finishing lip or band 130 of the open end 125 and with each fixable end 140 properly adhered to the surface of the sheath. FIG. 4 illustrates the proximal end view of the fitted sheath and shows the folds of material that gather at the underside of the open end 125 when the restraining strap 135 is applied. FIG. 5 illustrates that this embodiment of the sheath 100 can optionally be rolled for storage or application. The sheath 100 can be packaged, rolled or unrolled, in a plastic or foil pouch for storage, sales and distribution.

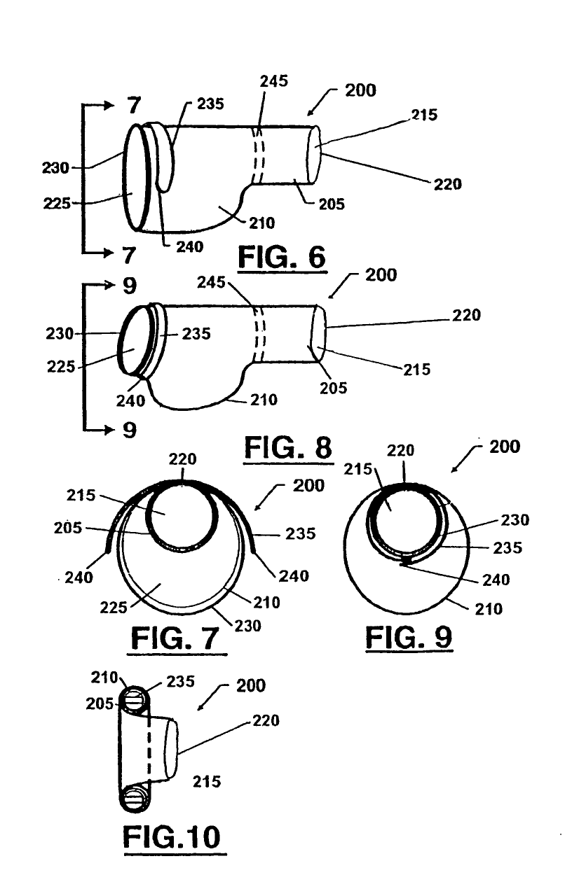

FIGS. 6 through 10 illustrate a second preferred embodiment of the present invention, which is a semi-fitted version of a sheath 200 configured to cover a portion of the user's penis and entire scrotum. FIG. 6 is a side view of the semi-fitted sheath 200 in an unfastened state. FIG. 7 is a proximal end view of the semi-fitted sheath 200 of FIG. 6 in the unfastened state. The semi-fitted sheath 200 is constructed with a roughly tubular shaft 205 that is attached at its proximal end to a scrotal sac 210, and with a distal end 215 and an open proximal end 225, similar to the fitted version previously described. However, in this embodiment, the scrotal sac 210 fits more relaxed and is less form-fitting around the scrotum, hence the designation semi-fitted. In this embodiment, the proximal open end 225 of the semi-fitted sheath 200 preferably represents the maximum diameter of the scrotal sac 210, and is significantly larger than the diameter of the tubular shaft 205. The scrotal sac 210 is asymmetrical with the tubular shaft 205 in keeping with the normal anatomical configuration of the male genitalia. When properly fitted, the tubular shaft 205 covers the lower portion penis and the scrotal sac 210 covers the entire scrotum.

Preferably, the sheath 200 is made of a highly flexible and elastic polymer or elastomer, including, but not limited to latex, polyurethane and other natural and synthetic rubbers. Preferably, the tubular shaft 205 and the scrotal sac 210 of the sheath 200 are manufactured together in one piece, such as by dip molding, out of a single material. Alternatively, however, the tubular shaft 205 and the scrotal sac 210 may be made of different materials and joined together, for instance by adhesive bonding or by heat welding. Additionally or alternatively, natural membranes or textile fabrics may be used in the construction of the tubular shaft 205 and/or scrotal sac 210 of the sheath 200.

The semi-fitted sheath 200 also includes a restraining strap 235 that, when fastened, secures the scrotum within the scrotal sac 210 and effectively anchors the sheath 200 during use. FIGS. 6 and 7 illustrate the restraining strap 235 in position, but not fastened. The restraining strap 235 is preferably constructed of a relatively flat elastic material and has fixable ends 240. The fixable ends 240 of the restraining strap 235 include a fastener, such as corresponding halves of a VELCRO hook-and-loop fastener strip, or an adhesive. In this illustrative embodiment, the restraining strap 235 is shown as permanently attached to the sheath 200 near the top of the open end 225. Optionally, however, the restraining strap 235 may be removably attached or completely separate from the sheath 200.

The periphery of the distal open end 220 is preferably finished with a lip or band 250 that is distinctly rounded and extends slightly above the surface of the tubular shaft 205 and that provides a useful gripping surface for helping to apply the sheath 200 over the surface of the penis. In one preferred embodiment, the lip or band 250 around the distal open end 225 of the penile shaft 220 is approximately 1 mm thick.

Optionally, the semi-fitted sheath 200 may also include a restrictive band 245, indicated by dashed lines in FIGS. 6 and 8, around the periphery of the tubular shaft 205. The restrictive band 245 is preferably located at the proximal end of the tubular shaft 205 where it joins to the scrotal sac 210. The restrictive band 245 may be formed by a slight thickening of the wall of the tubular shaft 205 and/or by a slight reduction in the internal diameter of the tubular shaft 205.

FIG. 8 is a side view of the semi-fitted sheath 200 of FIG. 6, shown in a fastened state. FIG. 9 is a proximal end view of the semi-fitted sheath 200 of FIG. 8 in the fastened state. When fastened, the restraining strap 235 with fixable ends 240 secures the scrotal sac 210 around the genitalia and anchors the sheath 200 during use. FIG. 10 illustrates the sheath 200 in a rolled condition for packaging and storage or for application. Other variations of the sheath may include rings, bands, ridges, bumps, and lumps on the interior or exterior surface of the sheath. Additionally or alternatively the sheath may have perforations, slits or messing to add ventilation.

Many features have been listed with particular configurations, options, and embodiments. Any one or more of the features described may be added to or combined with any of the other embodiments or other standard devices to create alternate combinations and embodiments.

Although the examples given include many specificities, they are intended as illustrative of only a few possible embodiments of the invention. Other embodiments and modifications will, no doubt, occur to those skilled in the art. Thus, the examples given should only be interpreted as illustrations of some of the preferred embodiments of the invention, and the full scope of the invention should be determined by the appended claims and their legal equivalents.

1. A condom configured to cover the user's scrotum and a portion of the user's penis, comprising:
a shaft portion,
a scrotum portion having one end thereof integrally formed with said shaft portion and another end thereof defining an opening,
and a strap member releasably disposed surrounding said opening of said scrotum portion.

2. The condom as set forth in claim 1, wherein said strap member comprises a strap having fixable ends with a hook-and-loop fastener for releasably fastening said strap around said opening of said scrotum portion.

3. The condom as set forth in claim 1, wherein said proximal opening of said scrotum portion includes a stopper ring integrally formed at and surrounding said opening, said stopper ring having a thickness substantially greater than the corresponding thickness of the material forming said scrotum portion.

4. The condom as set forth in claim 3, wherein said shaft portion, said scrotum portion and said stopper ring are formed of a material chosen from a group of materials consisting of polyurethane, natural lambskin, latex, plastic and rubber.

5. The condom as set forth in claim 3, wherein said strap member is a continuous electrometric band or an elastomeric band having fixable ends for releasably fastening said elastomeric band around said opening of said scrotum portion.

6. The condom as set forth in claim 1, wherein said shaft portion is eccentrically formed with said scrotum portion.

7. The condom as set forth in claim 1, wherein said shaft portion and said scrotum portion are configured to tightly fit onto the user.

8. The condom as set forth in claim 1, wherein said shaft portion and said scrotum portion are configured to loosely fit onto the user.

9. The condom as set forth in claim 1, wherein said shaft portion is configured to tightly fit onto the user and said scrotum portion is configured to loosely fit onto the user.

10. A condom configured to cover the user's scrotum and a portion of a user's penis, comprising:
a shaft portion having an open distal end,
a scrotum portion having one end thereof integrally formed with said open distal end of said shaft portion and the proximal end thereof defining an opening, said shaft portion including a stopper ring integrally formed at and surrounding said opening, said stopper ring having a thickness substantially greater than the corresponding thickness of the material forming said shaft portion, said scrotum portion having a different circumferential dimension than said shaft portion, said scrotum portion including a stopper ring integrally formed at and surrounding said opening, said stopper ring having a thickness substantially greater than the corresponding thickness of the material forming said scrotum portion,
and securing means encompassing said opening of said scrotum portion.

11. The condom as set forth in claim 10 wherein said securing means is an elastomeric band disposed at said opening adjacent to and retained by said stopper ring thereby preventing said elastomeric band from slipping beyond said opening of said scrotum portion.

12. The condom as set forth in claim 11, wherein said securing means comprises a strap having fixable ends with a hook-and-loop fastener for releasably fastening said strap around said opening of said scrotum portion.

13. The condom as set forth in claim 10, wherein said shaft portion and said scrotum portion are configured to tightly fit onto the user.

14. The condom as set forth in claim 11, wherein said shaft portion and said scrotum portion are configured to loosely fit onto the user.

15. The condom as set forth in claim 11, wherein said shaft portion is configured to tightly fit onto the user and said scrotum portion is configured to loosely fit onto the user.

16. The condom as set forth in claim 11, wherein said opening of said scrotum portion is sized to fit loosely around the user's scrotum and said securing means is configured to tighten said opening of said scrotum portion snugly onto the user.

17. The condom as set forth in claim 11, wherein said opening of said scrotum portion is of a diameter larger than said shaft portion.

18. The condom as set forth in claim 11, wherein said securing means comprises a strap having fixable ends for releasably fastening said strap around said opening of said scrotum portion.

19. The condom as set forth in claim 18, wherein said strap is permanently attached to the condom adjacent to said opening of said scrotum portion.
